(19) United States
(12) Patent Application Publication
(10) Pub. No.: US 2026/0224506 A1
(43) Pub. Date: Aug. 6, 2026

(54) MICRO-STRUCTURED EMULSIFICATION AND ENCAPSULATION OF BOTANICAL AND ORGANIC COMPOUNDS WITH GLYCEROL

(71) Applicant: 4EA, INC, TULSA, OK (US)

(72) Inventor: Paul Aston, Santee, CA (US)

(21) Appl. No.: 19/042,958

(22) Filed: Jan. 31, 2025

(51) Int. Cl. A61K 9/51; A61K 9/48

(52) U.S. Cl. CPC A61K 9/5192; A61K 9/4858

(57) ABSTRACT

This invention introduces a novel encapsulation technology for organic and botanical compounds, enhancing dissolution, stabilization, and bioavailability. The method dissolves hydrophobic botanical compounds in a weak organic acid (e.g., acetic, citric, or malic acid) to optimize solubility and molecular dispersion. A carefully balanced blend of mono-, di-, and triglycerides stabilizes the encapsulation matrix, preventing aggregation and ensuring uniformity. Incorporating a non-polar solvent enhances structural integrity, bioavailability, and absorption. High-shear mixing or ultrasonication further refines the encapsulation process, enabling broad applications across pharmaceuticals, cosmetics, functional foods, and veterinary products. This innovation enables controlled-release formulations, prolonged shelf life, and scalable production. By overcoming traditional challenges in hydrophobic compound delivery, this method revolutionizes encapsulation science, providing a sustainable and adaptable solution for industry-wide applications.

### BACKGROUND

### Field of Use

Within the realm of infused products, a prevalent practice entails the direct incorporation of organic compounds, botanicals or extracts into a wide array of consumer commodities, encompassing confections, beverages, pharmaceuticals, veterinary products and other consumable goods. Nevertheless, the outcomes of this traditional approach often fall short. This shortfall primarily stems from the distinctive attributes of plant-based botanicals, which predominantly consist of hydrocarbon structures. These hydrocarbon-based botanicals exhibit a lipid-like behavior, readily interacting with lipid-rich substances while displaying limited compatibility with aqueous solutions. Currently, it is difficult in the market for proprietors to provide the same structured organic compounds with a durable solution and formulation to stabilize the molecular interactions while providing durability for transport and storage, even over extended durations.

The fundamental rationale behind this behavior lies within the intricate electronic structure of molecules. Hydrocarbon structures, characterized by the near-equitable sharing of bonding electrons between carbon and hydrogen atoms, tend to preserve electrical neutrality. Consequently, there is minimal variation in the distribution of electric charge, or surface charge density, across such structures. These electrically neutral molecules tend to predominantly associate with like-minded neutral entities.

In stark contrast, water molecules exhibit both positively and negatively charged components due to unequal electron sharing between oxygen and hydrogen atoms. Oxygen's pronounced electron-attracting propensity results in the partial exposure of the positively charged nuclei of hydrogen atoms. This uneven electron distribution furnishes water molecules with both positive and negative charge regions, enabling interactions with other charged molecular species while precluding interaction with neutral counterparts. This fundamental discrepancy between hydrocarbon structures and water molecules underscores the longstanding challenge of achieving compatibility between oil and water, stemming from the absence of electronic interactions between their constituents.

### Background of Prior Art

Conventional concentrate production methods face challenges, particularly in the formulation of an innovative solution that transcends the limitations of conventional methods. The gravity of this challenge is underscored by the pervasive demand for products necessitating the incorporation of botanicals or extracts, spanning an array of industries, including pharmaceuticals, biotechnology, and consumer goods.

Historically, the production of foundational base stocks or formulations for these products has been beset by constraints, including limited versatility, scalability, and the capacity to incorporate an expansive repertoire of botanical and organic constituents. Moreover, ensuring the quality, purity, and uniformity of the resultant blends has proven to be a formidable hurdle.

In response to these pressing challenges, the present invention heralds a pioneering methodology poised to surmount the shortcomings of traditional processes. At its core, this groundbreaking methodology revolves around the encapsulation of organic or botanical compounds and related organic constituents within nano-scale composite particles meticulously engineered for rapid absorbability. This approach effectively circumvents the impediments associated with first-pass metabolism, thereby elevating the bioavailability and overall efficacy of infused products.

The Inventor has made many attempts at perfecting this design and it was not easy. It took Inventor several iterations of the method to fine tune nano-sizing and stabilization as well as constituent ratios and manufacturing costs. Each step of the process yielded insight as to how this method could be made cost-effectively and still reach the desired level of performance necessary.

### DESCRIPTION

It is imperative to note that the terminology employed herein is exclusively for the purpose of delineating specific embodiments and is not intended to impose limitations on the invention. The term “and/or” encompasses any and all conceivable combinations of one or more of the associated items. Furthermore, singular forms such as “a,” “an,” and “the” encompass plural forms as well as singular forms unless the context clearly dictates otherwise. The terms “comprises” and “comprising,” when used in this context, signify the presence of the stipulated features, steps, operations, elements, and/or components, without excluding the presence or incorporation of other features, steps, operations, elements, components, and/or groups thereof.

It is essential to underscore that, unless expressly defined, all terms used herein, including technical and scientific terminology, possess the same meanings as those commonly understood by individuals with ordinary skill in the pertinent field. Such terms, when not explicitly defined herein, should be construed in a manner that aligns with their meanings in the context of the relevant art and the disclosure presented herein. The term “botanicals,” for instance, broadly encompasses plant-derived compounds, including essential oils, terpenes, flavonoids, and hydrophobic active ingredients, but excludes overly specific identification of proprietary blends or ratios. This deliberate abstraction allows for a wide application of the innovation, setting it apart from narrower, less adaptable methodologies. All interpretations aim to maintain technical accuracy and patentability.

In elucidating the invention, it is crucial to acknowledge that numerous techniques and steps are disclosed. Each of these techniques and steps offers intrinsic benefits and can be employed either in isolation or in conjunction with one or more of the other disclosed methodologies. Thus, for the sake of clarity, this description refrains from repetitively enumerating all potential combinations of individual steps unless such repetition serves an essential explanatory purpose. These techniques are modular, adaptable, and presented in a manner that emphasizes flexibility. For example, the flexible configurations of mechanical processes, component ratios, and system parameters allow seamless adaptation to a variety of industries and products. Nevertheless, it should be unequivocally understood that the specification and claims encompass the entire gamut of conceivable combinations and permutations of the disclosed methodologies, establishing this invention as a novel contribution

The innovation commences with the dissolution of botanicals in a weak organic acid-broadly encompassing acetic, citric, or malic acids-meticulously adhering to specified ratios selected for compatibility with the target compounds. Notably, hydrophobic botanical compounds, particularly those featuring discrete carbon chain molecules, exhibit a pronounced affinity for cohesion. To address this inherent characteristic, the rendering and blending process, employing the organic acid, are leveraged to curtail the kinematic viscosity of these compounds. Critical steps, including control of acid-to-botanical ratios, enhance solubilization and molecular dispersion. The resulting viscosity reduction facilitates their swift mobility during the dynamic infusion process, culminating in comprehensive encapsulation. This precise configuration demonstrates innovation by overcoming historical limitations in the dissolution of hydrophobic compounds.

To augment the encapsulation process, a preordained proportion of mono-, di-, and triglycerides is incorporated into the solution. These glyceride chains serve a dual function: they interact both with the organic acid and the botanical compounds, while alternate segments of these molecules engage with the aqueous fluid. Each glyceride type is broadly discussed to emphasize functionality. This balance of glycerides, curated to support encapsulation success for each specific botanical, creates a robust emulsification, ensuring homogeneity and bioavailability. By utilizing an innovative interplay of these glycerides, this process distinguishes itself from conventional encapsulation techniques that fail to achieve comparable stability and absorption.

Hydrophobic botanical compounds-characterized by carbon chain molecules and categorized as fats-pose challenges addressed through precise rendering and blending processes. The incorporation of fats in the form of medium-chain, short-chain, and long-chain glycerides stabilizes these compounds. These glycerides act as intermediaries, ensuring stability and preventing aggregation during encapsulation. This novel application of glycerides for enhanced dispersion highlights the unique technical contributions of the invention. Physical methods such as ultrasonication or high-shear mixing are described in generalized terms, ensuring clarity.

As an integral facet of this innovative approach, trace amounts of non-polar solvent are included as an auxiliary component in the encapsulation process. By integrating a non-polar solvent in conjunction with glycerides, the encapsulation process achieves an exceptional level of stability and bioavailability. The resulting encapsulation matrix ensures stability, bioavailability, and compatibility with various applications that is bespoke to the specific botanical or other target for encapsulation. The disclosure emphasizes outcomes, such as enhanced solubility, stability, bioavailability, and absorption. This innovative matrix addresses significant industry challenges in delivering hydrophobic compounds, establishing this invention as a paradigm-shifting technology.

This pioneering encapsulation technique harbors immense potential for revolutionizing the landscape of infused products. Its applicability spans a diverse spectrum of fields, encompassing pharmaceuticals, cosmetics, functional foods, veterinary products and beyond. Examples include, respectively, encapsulation for controlled-release and enhanced bioavailability, stable formulations enabling improved skin penetration, infused consumables offering precise dosages and bioavailability, and tailored formulations for unique metabolic needs. A s research and development endeavors progress, the horizons of this groundbreaking methodology are poised to expand further, yielding innovative solutions and applications across multifarious domains.

Applications requiring specific adaptations (e.g., nanotechnology-based delivery or sustainable polymer systems) are described conceptually. By emphasizing adaptability, this invention positions itself as a cornerstone for future innovation across sectors.

This invention has several key technical advantages including significantly improved solubility and absorption of botanical compounds, modular ratios and components adaptable to various applications, prolonged shelf life through encapsulation and use of organic acids and natural glycerides that aligns with eco-conscious industry trends.

By emphasizing functional outcomes and general methodologies, this disclosure ensures technical clarity. The innovation uniquely addresses long-standing challenges in encapsulation science, marking a significant departure from traditional methods.

### DESCRIPTION OF THE DRAWINGS

Embodiments of the present invention are illustrated as an example and are not limited by the figures of the accompanying drawings, in which like references may indicate similar elements and in which:

FIG. 1 shows an embodiment of the present invention comprised of the following elements: a suite of glycerol compounds organized between a discrete layer of non-polar solvent and other glycerol compounds (1); a different formulation of glycerol compounds of varying molecular weights that rest and interact with the exterior facing glycerol compounds from 1 (2); a suite of varying glyceride compounds of different molecular weights that are contained within the glycerol wrapper (3); the trace non-polar solvent framework wrapper that surrounds the suite of glycerol, glyceride and both botanical and organic compounds (4); a suite of botanical and accompanying organic compounds are contained with the layer of glycerols, glycerides, and other wrapping components in a stable and molecularly discrete formation (5); and other organic molecules can also be incorporated into the organized compound as desired by the formulations as preferred by the operators (6).

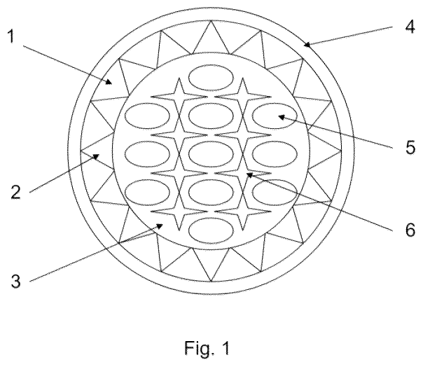

What is claimed is:

1. A method for encapsulating hydrophobic botanical compounds to enhance bioavailability, comprising: dissolving an at least one or more botanical compounds in an at least one or more proprietary weak organic acid solution under controlled conditions; introducing an at least one or more structured glyceride composition comprising a specific blend with an at least one or more unique structural properties; incorporating an at least one or more non-polar solvent compounds to optimize molecular interactions and facilitate structural cohesion; applying an at least one or more specialized mechanical processing protocol, including a proprietary sequence of high-shear mixing, to facilitate encapsulation; forming an at least one or more stabilized emulsion matrix capable of transmembrane transport with controlled-release properties.

2. The method of claim 1, wherein said proprietary weak organic acid solution is selected from a group consisting of acetic acid, citric acid, or malic acid, selected to maintain molecular integrity and prevent premature degradation of the encapsulated compounds.

3. The method of claim 1, wherein said proprietary weak organic acid is selected to maintain molecular integrity and prevent premature degradation of the encapsulated compounds.

4. The method of claim 1, wherein said non-polar solvent is selected from a group comprising plant-derived oils, medium-chain triglycerides, and pharmaceutically acceptable hydrocarbon solvents introduced at a calibrated phase to optimize encapsulation efficiency.

5. The method of claim 1, wherein said non-polar solvent component is introduced at a calibrated phase to optimize encapsulation efficiency without compromising compound stability.

6. The method of claim 1, wherein said at least one structured glyceride composition is selected to enhance molecular compatibility with the botanical compounds and improve encapsulation efficiency.

7. The method of claim 1, wherein said specialized mechanical processing protocol is adapted to control particle morphology, size distribution, and surface properties.

8. The method of claim 1, further comprising dynamically adjusting the ratio of said an at least one or more botanical compounds to glycerides using an adaptive formulation approach to achieve targeted release kinetics and prolonged shelf stability.

9. The method of claim 1, wherein said encapsulated hydrophobic botanical compounds demonstrate enhanced solubility and increased bioavailability compared to non-encapsulated botanical extracts.

10. The method of claim 1, wherein said emulsion matrix is formulated to withstand environmental stressors, including temperature fluctuations, oxidative degradation, and mechanical agitation during transport and storage.

11. The method of claim 1, further comprising incorporating an at least one or more emulsifiers to further improve absorption and compatibility with aqueous-based formulations.

12. The method of claim 1, further comprising incorporating an at least one or more bioenhancers to further improve absorption and compatibility with aqueous-based formulations.
